(19) United States
(12) Patent Application Publication
(10) Pub. No.: US 2018/0015014 A1
(43) Pub. Date: Jan. 18, 2018

(54) Mouthrinse Formulations

(71) Applicant: GABA International Holding GmbH, Therwil (CH)

(72) Inventors: Ruth HINRICHS, Therwil (CH); Turan MATUR, Binningen (CH); Andre BRUNELLA, Dornach (CH); Sylvia HESS, Holstein (CH)

(73) Assignee: GABA International Holding GmbH, Therwil (CH)

(21) Appl. No.: 15/529,680

(22) PCT Filed: Dec. 17, 2015
(86) PCT No.: PCT/US2015/066304

§ 371 Date: May 25, 2017

Related U.S. Application Data
(60) Provisional application No. 62/093,545, filed on Dec. 18, 2014.

(51) Int. Cl. A61K 8/362; A61K 8/21; A61K 8/41; A61Q 11/00

(52) U.S. Cl. CPC A61K 8/362; A61Q 11/00; A61K 8/21; A61K 8/416; A61K 2800/52

(57) ABSTRACT

An oral care mouth rinse composition containing at least one source of ionic tin and at least one aliphatic di- or tri-carboxylic acid in free or salt form, wherein the at least one aliphatic di- or tri-carboxylic acid in free or salt form is present in an amount of from 0.05 to 0.30 weight % based on the total weight of the oral care composition is provided.

The present invention relates to oral care compositions comprising ionic tin and in particular to stannous-containing mouth rinse formulations.

### BACKGROUND

Tin ions such as stannous ions have been incorporated into oral care compositions including dentifrices and mouth rinses in order to provide anti-caries benefits and in order to treat and/or prevent gingivitis. However, mouth rinse formulations comprising tin ions can be unstable and can have a tendency to form a white precipitate upon storage. This is the result of the formation of the highly insoluble salt stannic oxide, which precipitates from the formulation.

It would be desirable to be able to formulate stable oral care mouth rinse compositions comprising tin ions.

### BRIEF SUMMARY

It has now surprisingly been discovered that the combination of tin ions with 0.05 to 0.30 weight % aliphatic di- or tri-carboxylic acid in a mouth rinse provides a highly stable oral care mouth rinse formulation that combines oral care benefits with storage stability.

According to a first aspect of the present invention there is provided an oral care mouth rinse composition comprising:

at least one source of ionic tin and

at least one aliphatic di- or tri-carboxylic acid in free or salt form

wherein the at least one aliphatic di- or tri-carboxylic acid in free or salt form is present in an amount of from 0.05 to 0.30 weight % based on the total weight of the oral care composition.

Optionally the oral care mouth rinse composition comprises 0.10 to 0.25 weight % aliphatic di- or tri-carboxylic acid in free or salt form. Further optionally the oral care mouth rinse composition comprises 0.10 to 0.20 weight % aliphatic di- or tri-carboxylic acid in free or salt form. Further optionally the oral care mouth rinse composition comprises 0.12 to 0.15 weight % aliphatic di- or tri-carboxylic acid in free or salt form.

Optionally the at least one aliphatic di- or tri-carboxylic acid in free or salt form comprises a C₃ to C₇ aliphatic di-carboxylic acid in free or salt form. Further optionally the at least one aliphatic di- or tri-carboxylic acid comprises malic acid in free or salt form. Further optionally the oral care mouth rinse composition comprises 0.10 to 0.25 weight % malic acid. Further optionally the oral care mouth rinse composition comprises 0.10 to 0.20 weight % malic acid. Further optionally the oral care mouth rinse composition comprises 0.12 to 0.15 weight % malic acid.

Optionally the source of ionic tin is selected from the group comprising stannous ion sources, stannic ion sources and combinations thereof. Optionally the source of ionic tin is selected from the group comprising stannous fluoride, stannous chloride, stannic fluoride, stannic chloride, stannic acetate, stannous acetate and combinations thereof. Optionally the oral care mouth rinse composition comprises at least one stannous ion source. Optionally the source of ionic tin is selected from the group comprising stannous fluoride, stannous chloride, stannous acetate and combinations thereof.

Optionally the concentration of ionic tin is from 0.01 to 0.10 weight % based on the total weight of the oral care mouth rinse composition. Further optionally the concentration of ionic tin is from 0.02 to 0.08 weight % based on the total weight of the oral care mouth rinse composition. Further optionally the concentration of ionic tin is from 0.03 to 0.06 weight % based on the total weight of the oral care mouth rinse composition. Further optionally the concentration of ionic tin is 0.04 weight % based on the total weight of the oral care mouth rinse composition.

Optionally the at least one source of ionic tin comprises stannous fluoride.

Optionally the oral care mouth rinse composition further comprises a non-stannous fluoride ion source. Further optionally the oral care mouth rinse composition comprises sodium fluoride, potassium fluoride, sodium monofluorophosphate, sodium fluorosilicate, ammonium fluorosilicate, amine fluoride, ammonium fluoride and combinations thereof. Further optionally the oral care mouth rinse composition comprises sodium fluoride or amine fluoride. Further optionally the oral care mouth rinse composition comprises amine fluoride or sodium fluoride in an amount corresponding to 45 to 1500 ppm fluoride based on the total weight of the composition. Further optionally the oral care mouth rinse composition comprises 0.06 to 0.33 weight % amine fluoride or 0.01 to 0.33 weight % sodium fluoride or a combination thereof, based on the total weight of the composition.

Optionally the oral care mouth rinse composition comprises 5 to 1500 ppm fluoride.

Optionally the oral care mouth rinse composition comprises 0.10 to 0.25 weight % malic acid, 0.03 to 0.08 weight % stannous fluoride and 0.10 to 0.25 weight % amine fluoride.

Optionally the oral care mouth rinse composition comprises 0.10 to 0.25 weight % malic acid, 0.04 to 0.08 weight % stannous fluoride and 0.10 to 0.25 weight % N,N,N′-tris(2-hydroxyethyl)-N′-octadecyl-1,3-diaminopropanedihydrofluoride.

According to a further aspect of the invention there is provided a method to reduce or inhibit formation of dental caries, reduce, repair or inhibit pre-carious lesions of the enamel, reduce or inhibit demineralization and promote remineralization of the teeth reduce hypersensitivity of the teeth, reduce or inhibit gingivitis, promote healing of sores or cuts in the oral cavity, reduce levels of acid producing bacteria, reduce or inhibit microbial biofilm formation in the oral cavity, reduce or inhibit plaque formation in the oral cavity, promote systemic health and/or clean teeth and oral cavity, comprising applying an effective amount of an oral care mouth rinse composition as described herein to the oral cavity of a subject in need thereof.

According to a further aspect of the present invention there is provided an oral care mouth rinse composition for use in a method to reduce or inhibit formation of dental caries, reduce, repair or inhibit pre-carious lesions of the enamel, reduce or inhibit demineralization and promote remineralization of the teeth, reduce hypersensitivity of the teeth, reduce or inhibit gingivitis, promote healing of sores or cuts in the oral cavity, reduce levels of acid producing bacteria, reduce or inhibit microbial biofilm formation in the oral cavity, reduce or inhibit plaque formation in the oral cavity, promote systemic health clean teeth and oral cavity.

According to a further aspect of the present invention there is provided use of an oral care mouth rinse composition to reduce or inhibit formation of dental caries, reduce, repair or inhibit pre-carious lesions of the enamel, reduce or inhibit demineralization and promote remineralization of the teeth, reduce hypersensitivity of the teeth, reduce or inhibit gingivitis, promote healing of sores or cuts in the oral cavity, reduce levels of acid producing bacteria, reduce or inhibit microbial biofilm formation in the oral cavity, reduce or inhibit plaque formation in the oral cavity, promote systemic health and/or clean teeth and oral cavity in a subject in need thereof.

According to a further aspect of the present invention there is also provided a method of stabilizing ionic tin in an oral care mouth rinse composition comprising at least one source of ionic tin, wherein the method comprises formulating the oral care mouth rinse composition to comprise at least one aliphatic di- or tri-carboxylic acid in free or salt form in an amount of from 0.05 to 0.30 weight % based on the total weight of the oral care composition. Optionally the method comprises formulating the oral care mouth rinse composition to comprise from 0.05 to 0.30 weight % malic acid in free or salt form. Optionally the concentration of ionic tin in the oral care mouth rinse composition is from 0.01 to 0.10 weight % based on the total weight of the oral care mouth rinse composition. Optionally the composition further comprises 0.10 to 0.25 weight % amine fluoride.

According to a further aspect of the present invention there is also provided a method of reducing staining of surfaces in the oral cavity resulting from use of an oral care mouth rinse comprising at least one source of ionic tin, wherein the method comprises formulating the oral care mouth rinse composition to comprise at least one aliphatic di- or tri-carboxylic acid in free or salt form in an amount of from 0.05 to 0.30 weight % based on the total weight of the oral care composition. Optionally the method comprises formulating the oral care mouth rinse composition to comprise from 0.05 to 0.30 weight % malic acid in free or salt form. Optionally the concentration of ionic tin in the oral care mouth rinse composition is from 0.01 to 0.10 weight % based on the total weight of the oral care mouth rinse composition. Optionally the composition further comprises 0.10 to 0.25 weight % amine fluoride.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses. The composition may include a first feature described in one example composition herein, as well as a second feature described in another example. In other words, the invention contemplates mixing and matching features from the disclosed embodiments in various combinations.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

It has surprisingly been found that aliphatic di- or tri-carboxylic acids can be used in tin-containing mouth rinse formulations to improve the stability of the ionic tin. The inventors of the present application have discovered that such acids can reduce or prevent the formation of insoluble precipitate and/or a turbid appearance upon storage and ageing. Previously, mouth rinse formulations comprising ionic tin have had a tendency to form sediments and/or develop a turbid appearance when stored, for example at 25° C. for three or more months.

Surprisingly, formulations comprising aliphatic di- or tri-carboxylic acids also showed a reduction in staining. Previously, compositions comprising ionic tin have shown a tendency to stain teeth in vivo.

Mouth rinse (or mouthwash) compositions comprise one or more oral care active component in a liquid carrier. Typically, mouth rinse compositions are formulated suing a solvent such as water. They may be used to deliver oral care actives to the oral cavity of a consumer and may be used either before or after brushing and/or flossing.

The mouth rinse compositions of the present invention comprise a source of ionic tin. In certain embodiments the source of ionic tin comprises a stannous or stannic salt, or a combination thereof. In certain embodiments the source of ionic tin comprises a stannous salt. In certain embodiments the source of ionic tin is selected from water-soluble stannous salts such as stannous fluoride, stannous chloride, stannous acetate and combinations thereof. In certain embodiments the source of ionic tin comprises stannous fluoride, stannous chloride or combinations thereof. In certain preferred embodiments, the source of ionic tin is stannous fluoride. In certain embodiments separate soluble stannous and fluoride salts may be used to provide stannous fluoride in situ. Alternatively, stannous fluoride salt may be added to the composition directly.

In one embodiment, the present invention provides an oral mouth rinse composition (Composition 1) comprising: (i) at least one source of ionic tin and (ii) at least one aliphatic di- or tri-carboxylic acid in free or salt form, wherein the at least one aliphatic di- or tri-carboxylic acid in free or salt form is present in an amount of from 0.05 to 0.30 weight % based on the total weight of the oral care composition. In further embodiments, the present invention provides Compositions as follows:
1.1 Composition 1, comprising 0.10 to 0.25 weight % aliphatic di- or tri-carboxylic acid in free or salt form.
1.2 Composition 1 or 1.1, comprising 0.10 to 0.20 weight % aliphatic di- or tri-carboxylic acid in free or salt form.
1.3 Composition 1, 1.1 or 1.2, comprising 0.12 to 0.15 weight % aliphatic di- or tri-carboxylic acid in free or salt form.
1.4 Composition 1, wherein the at least one aliphatic di- or tri-carboxylic acid in free or salt form comprises a C3 to C7 aliphatic di-carboxylic acid in free or salt form.
1.5 Composition 1 or 1.1 et seq. wherein the at least one aliphatic di- or tri-carboxylic acid comprises malic acid in free or salt form.
1.6 Composition 1 or 1.1 et seq. comprising 0.10 to 0.25 weight % malic acid.
1.7 Composition 1 or 1.1 et seq. comprising 0.10 to 0.20 weight % malic acid.
1.8 Composition 1 or 1.1 et seq. comprising 0.12 to 0.15 weight % malic acid.
1.9 Composition 1 or 1.1 et seq. wherein the source of ionic tin is selected from the group comprising stannous ion sources, stannic ion sources and combinations thereof.
1.10 Composition 1 or 1.1 et seq. wherein the source of ionic tin is selected from the group comprising stannous fluoride, stannous chloride, stannic fluoride, stannic chloride, stannic acetate, stannous acetate and combinations thereof.
1.11 Composition 1 or 1.1 et seq. wherein the composition comprises at least one stannous ion source.
1.12 Composition 1 or 1.1 et seq. wherein the source of ionic tin is selected from the group comprising stannous fluoride, stannous chloride, stannous acetate and combinations thereof.
1.13 Composition 1 or 1.1 et seq. wherein the concentration of ionic tin is from 0.01 to 0.10 weight % based on the total weight of the oral care mouth rinse composition.
1.14 Composition 1 or 1.1 et seq. wherein the concentration of ionic tin is from 0.02 to 0.08 weight % based on the total weight of the oral care mouth rinse composition.
1.15 Composition 1 or 1.1 et seq. wherein the concentration of ionic tin is from 0.03 to 0.06 weight % based on the total weight of the oral care mouth rinse composition.
1.16 Composition 1 or 1.1 et seq. wherein the concentration of ionic tin is 0.04 weight % based on the total weight of the oral care mouth rinse composition.
1.17 Composition 1 or 1.1 et seq. wherein the source of ionic tin comprises stannous fluoride.
1.18 Composition 1 or 1.1 et seq. further comprising a non-stannous fluoride ion source.
1.19 Composition 1 or 1.1 et seq. comprising sodium fluoride, potassium fluoride, sodium monofluorophosphate, sodium fluorosilicate, ammonium fluorosilicate, amine fluoride, ammonium fluoride and combinations thereof.
1.20 Composition 1 or 1.1 et seq. comprising sodium fluoride or amine fluoride.
1.21 Composition 1 or 1.1 et seq. comprising amine fluoride or sodium fluoride in an amount corresponding to 45 to 1500 ppm fluoride based on the total weight of the composition.
1.22 Composition 1 or 1.1 et seq. comprising 0.06 to 0.33 weight % amine fluoride or 0.01 to 0.33 weight % sodium fluoride or a combination thereof based on the total weight of the composition.
1.23 Composition 1 or 1.1 et seq. comprising 5 to 1500 ppm fluoride.
1.24 Composition 1 or 1.1 et seq. comprising 0.10 to 0.25 weight % malic acid, 0.04 to 0.08 weight % stannous fluoride and 0.10 to 0.25 weight % N,N,N′-tris(2-hydroxyethyl)-N′-octadecyl-1,3-diaminopropane dihydrofluoride.
1.25 Composition 1 or 1.1 et seq. comprising 0.10 to 0.25 weight % malic acid, 0.03 to 0.08 weight % stannous fluoride and 0.10 to 0.25 weight % N,N,N′-tris(2-hydroxyethyl)-N′-octadecyl-1,3-diaminopropane dihydrofluoride.
1.26 Composition 1 or 1.1 et seq., wherein the composition comprises an amine fluoride.
1.27 Composition 1.26, wherein the composition comprises an amine fluoride in an amount of from 0.10 to 0.25% by weight of the composition.
1.28 Composition 1.26 or 1.27, wherein the amine fluoride is selected from N,N,N′-tris(2-hydroxyethyl)-N′-octadecyl-1,3-diaminopropane dihydrofluoride, 9-octadecenylamine hydrofluoride, or a combination thereof.
1.29 Composition 1.26 or 1.27 wherein the amine fluoride is a mixture of N,N,N′-tris(2-hydroxyethyl)-N′-octadecyl-1,3-diaminopropane dihydrofluoride, and 9-octadecenylamine hydrofluoride.
1.30 Any of Compositions 1 or 1.1 to 1.25, or any of Compositions 1.26-1.29, for use in a method to:
   (i) reduce or inhibit formation of dental caries,
   (ii) reduce, repair or inhibit pre-carious lesions of the enamel,
   (iii) reduce or inhibit demineralization and promote remineralization of the teeth, (iv) reduce hypersensitivity of the teeth,
   (v) reduce or inhibit gingivitis,
   (vi) promote healing of sores or cuts in the oral cavity,
   (vii) reduce levels of acid producing bacteria,
   (viii) reduce or inhibit microbial biofilm formation in the oral cavity,
   (ix) reduce or inhibit plaque formation in the oral cavity,
   (x) promote systemic health, or
   (xi) clean teeth and oral cavity.

In certain embodiments the concentration of ionic tin in the oral care mouth rinse composition is from 0.01 to 0.10 weight % based on the total weight of the oral care mouth rinse composition. In certain embodiments the concentration of ionic tin in the oral care mouth rinse composition is from 0.02 to 0.08 weight % or from 0.03 to 0.06 weight %. In certain embodiments the concentration of ionic tin is about 0.040 weight % based on the total weight of the oral care mouth rinse composition.

The compositions of the present invention comprise at least one aliphatic di- or tri-carboxylic acid in free or salt form. In certain embodiments the carboxylic acid may be added as the free acid or as a salt. In certain embodiments, the carboxylic acid is added as the sodium or potassium salt. In certain embodiments the oral care mouth rinse composition comprises a C₃ to C₇ aliphatic di-carboxylic acid. In certain embodiments the carboxylic acid is an α-hydroxy acid. In certain embodiments the carboxylic acid is an α-hydroxy di-carboxylic acid. In certain embodiments the carboxylic acid is malic acid, a salt of malic acid, or a combination thereof. The carboxylic acid may be a racemic mixture of the L- and D-form of an aliphatic di- or tri-carboxylic acid. In certain embodiments the carboxylic acid is a racemic mixture of L-malic acid and D-malic acid.

The aliphatic di- or tri-carboxylic acid in free or salt form is present in an amount of from 0.05 to 0.30 weight % based on the total weight of the oral care composition. In certain embodiments the composition comprises 0.10 to 0.25 weight %, 0.10 to 0.20 weight %, 0.10 to 0.15 weight %, 0.12 to 0.25 weight %, 0.12 to 0.20, or 0.12 to 0.15 weight % aliphatic di- or tri-carboxylic acid in free or salt form. In certain embodiments the composition comprises 0.10 to 0.25 weight %, 0.10 to 0.20 weight %, 0.10 to 0.15 weight %, 0.12 to 0.25 weight %, 0.12 to 0.20, or 0.12 to 0.15 weight % malic acid. In certain embodiments the composition comprises 0.10 to 0.25 weight %, 0.10 to 0.20 weight %, 0.10 to 0.15 weight %, 0.12 to 0.25 weight %, 0.12 to 0.20, or 0.12 to 0.15 weight % malic acid.

In certain embodiments the mouth rinse composition comprises a non-stannous fluoride ion source i.e. a source of fluoride ions that is not a stannous salt. In certain embodiments the compositions of the invention comprise sodium fluoride, potassium fluoride, sodium monofluorophosphate, sodium fluorosilicate, ammonium fluorosilicate, amine fluoride, ammonium fluoride and/or combinations thereof. In certain embodiments the composition comprises a fluoride ion source in a concentration sufficient to supply about 5 ppm to about 1,500 ppm fluoride ions, for example from about 25 ppm to about 1,500 ppm, from about 100 ppm to about 1,500 ppm, from about 200 ppm to about 550 ppm or from about 250 ppm to about 500 ppm.

In certain embodiments, the mouth rinse composition comprises sodium fluoride, amine fluoride and/or a combination thereof. In certain embodiments the composition comprises OLAFLUR (N,N,N′-tris(2-hydroxyethyl)-N′-octadecyl-1,3-diaminopropane dihydrofluoride) or DECTAFLUR (9-octadecenylaminehydrofluoride) in an amount of from 0.10 to 0.25 weight % or a mixture of both OLAFLUR and DECTAFLUR.

In certain embodiments, the mouth rinse composition comprises a combination of a source of ionic tin and amine fluoride, for example a stannous salt and amine fluoride. In certain embodiments the mouth rinse composition comprises stannous fluoride and amine fluoride. In certain embodiments the mouth rinse composition comprises stannous fluoride and OLAFLUR (N,N,N′-tris(2-hydroxyethyl)-N′-octadecyl-1,3-diaminopropanedihydrofluoride).

In certain embodiments the compositions of the invention may also comprise a salt of pyrrolidone carboxylic acid (also known as PCA, 2-pyrrolidone-5-carboxylic acid; 5-oxoproline; pidolic acid or pyroglutamic acid). In certain embodiments the compositions of the invention may comprise a salt of the L-enantiomer of pyrrolidone carboxylic acid, for example zinc pyrrolidone carboxylate, copper pyrrolidone carboxylate, magnesium pyrrolidone carboxylate or manganese pyrrolidone carboxylate. In certain embodiments the compositions of the invention may comprise zinc L-pyrrolidone carboxylate. The salt of pyrrolidone carboxylic acid may be present in an amount of from 0.01 to 0.75 weight %, for example from 0.05 to 0.50 weight % or from 0.10 to 0.20 weight.

In any of the above embodiments, the compositions may further comprise a flavoring agent. The flavoring agent may comprise one or more essential oils as well as various flavoring aldehydes, esters and/or alcohols. In certain embodiments, the flavoring agent comprises one or more essential oil selected form oils of peppermint, wintergreen, sassafras, clove, sage, eucalyptus, marjoram, cinnamon, lemon, lime, grapefruit and orange. In certain embodiments, the flavoring agent comprises oils of peppermint and spearmint. In certain embodiments the composition comprises from 0.01 to 2.0 weight %, 0.05 to 1.0 weight % or 0.10 to 0.20 weight % flavoring agent based on the total weight of the composition.

In any of the above embodiments, the compositions may further comprise at least one surfactant. Any orally acceptable surfactant such as a nonionic, anionic or amphoteric surfactant may be used. Optionally a surfactant may be present in an amount of from 0.01 weight % to 10 weight %, for example from 0.05 to 5 weight % or from 0.10 to 2.0 weight % based on the total weight of the composition.

In any of the above embodiments, the compositions may further comprise a sweetener such as, for example, sodium saccharin. One or more sweeteners may be present in an amount of from 0.005 weight % to 5 weight % by total weight of the composition, for example, 0.05 weight % to 0.1 weight %.

In any of the above embodiments, the compositions may further comprise at least one colorant. Colorants may include pigments, dyes, lakes and agents imparting a particular luster or reflectivity such as pearling agents. Any orally acceptable colorant can be used. One or more colorants may optionally be present in the compositions in an amount of from 0.0001 weight % to 20 weight %, for example from 0.0001 weight % to 1 weight % or from about 0.0001 weight % to 0.010 weight %.

In any of the above embodiments, the compositions may further comprise a polyhydric alcohol such as glycerin, sorbitol, xylitol, propylene glycol and combinations thereof. In certain embodiments the compositions may optionally comprise form about 0.10 to about 10 weight % polyhydric alcohol based on the total weight of the composition. In certain embodiments the compositions may comprise from 0.50 to 10 weight % xylitol, for example from 0.50 to 7.0 weight % xylitol. In certain embodiments the compositions may comprise from 0.50 to 5.0 weight % glycerin, for example 1.0 to 3.5 weight % glycerin. In certain embodiments the compositions may comprise 0.10 to 1.0 weight % propylene glycol. In any of the above embodiments, the compositions may comprise 0.50 to 7.0 weight % xylitol and 1.0 to 3.5 weight % glycerin.

In any of the above embodiments, the compositions may further comprise one or more polymers such as polyethylene glycols, polyvinylmethyl ether maleic acid copolymers and polysaccharides (e.g. cellulose derivatives such as carboxymethyl cellulose or microcrystalline cellulose, or polysaccharide gums such as xanthan gum or carrageenan gum). Such polymers may be present in any of the compositions of the invention in an amount of from 0.05 to 5.0 weight %. In any of the above embodiments, the compositions may further comprise a pH adjuster. For example the compositions may comprise an acid or base in an amount sufficient to adjust the pH of the compositions such that the compositions have a pH of from 4.0 to 8.0. In certain embodiments, the compositions may include aqueous potassium hydroxide as a pH adjuster. In certain embodiments the compositions may include aqueous potassium hydroxide in an amount such that the pH of the composition is from 5.5 to 7.5. In certain embodiments aqueous potassium hydroxide is added to the compositions to adjust the pH to around 6.5 to 7.5, for example about 7.0.

In another aspect, the present invention provides a method (Method 1) of treating or preventing diseases or disorders of the oral cavity of a subject in need thereof, the method comprising applying an effective amount of an oral care mouth rinse composition of the present invention, e.g., Composition 1 or any of 1.1-1.25, or any of Composition 1.26-1.29.

In a further embodiment, the present invention provides a method (Method 1.1) according to Method 1, wherein the method is effective to:
(i) reduce or inhibit formation of dental caries,
(ii) reduce, repair or inhibit pre-carious lesions of the enamel,
(iii) reduce or inhibit demineralization and promote remineralization of the teeth,
(iv) reduce hypersensitivity of the teeth,
(v) reduce or inhibit gingivitis,
(vi) promote healing of sores or cuts in the oral cavity,
(vii) reduce levels of acid producing bacteria,
(viii) reduce or inhibit microbial biofilm formation in the oral cavity,
(ix) reduce or inhibit plaque formation in the oral cavity,
(x) promote systemic health, or
(xi) clean teeth and oral cavity;
   and the method comprises applying an effective amount of an oral care mouth rinse composition according to any preceding claim to the oral cavity of a subject in need thereof.

In a further aspect, the present invention provides use of an oral care mouth rinse composition of the present invention, e.g., Composition 1 or any of 1.1-1.25, or any of Composition 1.26-1.29 to:
(i) reduce or inhibit formation of dental caries,
(ii) reduce, repair or inhibit pre-carious lesions of the enamel,
(iii) reduce or inhibit demineralization and promote remineralization of the teeth,
(iv) reduce hypersensitivity of the teeth,
(v) reduce or inhibit gingivitis,
(vi) promote healing of sores or cuts in the oral cavity,
(vii) reduce levels of acid producing bacteria,
(viii) reduce or inhibit microbial biofilm formation in the oral cavity,
(ix) reduce or inhibit plaque formation in the oral cavity,
(x) promote systemic health, or
(xi) clean teeth and oral cavity

in a subject in need thereof.

In another aspect, the present invention provides s method (Method 2) of stabilizing ionic tin in an oral care mouth rinse composition comprising at least one source of ionic tin, wherein the method comprises formulating the oral care mouth rinse composition to comprise at least one aliphatic di- or tri-carboxylic acid in free or salt form in an amount of from 0.05 to 0.30 weight % based on the total weight of the oral care composition. In further embodiments the present invention provides Methods as follows:
2.1 Method 2, wherein the method comprises formulating the oral care mouth rinse composition to comprise from 0.05 to 0.30 weight % malic acid in free or salt form.
2.2 Method 2 or 2.1, wherein the concentration of ionic tin in the oral care mouth rinse composition is from 0.01 to 0.10 weight % based on the total weight of the oral care mouth rinse composition.
2.3 Method 2 or 2.1 et seq. wherein the composition further comprises 0.10 to 0.25 weight % amine fluoride.
2.4 Method 2.3, wherein the amine fluoride is selected from N,N,N′-tris(2-hydroxyethyl)-N′-octadecyl-1,3-diaminopropane dihydrofluoride, 9-octadecenylamine hydrofluoride, or a combination thereof.
2.5 Method 2.3, wherein the amine fluoride is a mixture of N,N,N′-tris(2-hydroxyethyl)-N′-octadecyl-1,3-diaminopropane dihydrofluoride, and 9-octadecenylamine hydrofluoride.

In another aspect, the present invention provides s method (Method 3) of reducing staining of surfaces in the oral cavity resulting from use of an oral care mouth rinse comprising at least one source of ionic tin, wherein the method comprises formulating the oral care mouth rinse composition to comprise at least one aliphatic di- or tri-carboxylic acid in free or salt form in an amount of from 0.05 to 0.30 weight % based on the total weight of the oral care composition.

In a further embodiment, the present invention provides methods as follows:
3.1 Method 3, wherein the method comprises formulating the oral care mouth rinse composition to comprise from 0.05 to 0.30 weight % malic acid in free or salt form.
3.2 Method 3 or 3.1, wherein the concentration of ionic tin in the oral care mouth rinse composition is from 0.01 to 0.10 weight % based on the total weight of the oral care mouth rinse composition.
3.3 Method 3, 3.1 or 3.2, wherein the composition further comprises 0.10 to 0.25 weight % amine fluoride.
3.4 Method 3.3, wherein the amine fluoride is selected from N,N,N′-tris(2-hydroxyethyl)-N′-octadecyl-1,3-diaminopropane dihydrofluoride, 9-octadecenylamine hydrofluoride, or a combination thereof.
3.5 Method 3.3, wherein the amine fluoride is a mixture of N,N,N′-tris(2-hydroxyethyl)-N′-octadecyl-1,3-diaminopropane dihydrofluoride, and 9-octadecenylamine hydrofluoride.

### EXAMPLES

### Example 1

Mouth rinse formulations are prepared by mixing water, powdered ingredients and colorants in a vessel and stirring to dissolve. Stannous fluoride is dissolved in concentrated amine fluoride solution and added to the vessel. This mixture is stirred until homogeneous. Aroma, surfactants and all other remaining ingredients are added and the mixture stirred until the solution is clear. If necessary, the pH is adjusted.

Mouth rinse formulations A and B (Table 1) were prepared and evaluated in terms of their appearance and texture after ageing by storage at either 25° C. or 40° C.

TABLE 1

| | Formulation A | Formulation B |
|---|---|---|
| Malic acid | 0.00 | 0.20 |
| Xylitol | 0.85 | 0.85 |
| PVP (MW 58,000) | 0.30 | 0.30 |
| Emulsifier | 0.25 | 0.25 |
| Stannous fluoride | 0.05 | 0.05 |
| Amine fluoride | 0.17 | 0.17 |
| Water, sweetener, aroma, | QS | QS |
| color, pH adjuster | | |

The formulations were evaluated using a 4 point scale from 4 (best) to 1 (fail).

TABLE 2

| | Appearance | Appearance | Appearance | Appearance |
|---|---|---|---|---|
| | (color) | (color) | (color) | (color) after |
| | after 3 months | after 3 months | after 6 months | 6 months |
| Formula | at 25° C. | at 40° C. | at 25° C. | at 40° C. |
| A | Pass (3) | Pass (3) | Pass (3) | Pass (3) |
| | Light blue | Light blue | Light blue | Light blue |
| | solution | solution | solution | solution with |
| | | | | green shade |
| B | Pass (3) | Pass (3) | Pass (4) | Pass (4) |
| | Light blue | Light blue | | |
| | solution | solution | | |

TABLE 3

| | Appearance (texture) | Appearance (texture) | Appearance (texture) | Appearance (texture) |
|---|---|---|---|---|
| Formula | after 3 months at 25° C. | after 3 months at 40° C. | after 6 months at 25° C. | after 6 months at 40° C. |
| A | Pass (3) | Pass (2) | Pass(2) | Fail (1) |
| | Slightly turbid solution | Slightly turbid solution | Slightly turbid solution | Almost turbid solution |
| | with traces of sediments | with some sediments | with slight sediments | with some sediments |
| B | Pass (3) | Pass (3) | Pass (4) | Pass (4) |
| | Borderline Clear solution | Borderline Clear solution | Clear solution without | Clear solution without |
| | without sediments | without sediments | sediments | sediments |

These results demonstrate that addition of malic acid to a stannous containing mouth rinse reduces the formation of turbidity and reduce sediment formation. The malic acid containing formulations stay clear without sediment even after accelerated ageing at 40° C. for 6 months.

### Example 2

Additional mouthrinse formulations C through H are similarly compared as to color and texture during accelerated aging at 40° C. for 6 months. The results are shown in Table 3. Each formulation C through H has the same composition as formulation B, above, except for the malic acid content. It is found that all six of these additional formulations remain light blue solutions throughout the 6-months, however, it is found that a malic acid content of from 0.3 to 0.5% results in a clear solution at 6-months, but 0.05-0.1% malic acid does not.

TABLE 4

| | Appearance (texture) | Appearance (texture) |
|---|---|---|
| Formula | after 3 months at 40° C. | after 6 months at 40° C. |
| C | Pass (2) | Fail (1) |
| (0.01% | Slightly turbid solution | Turbid solution |
| Malic Acid) | | |
| D | Pass (2) | Fail (1) |
| (0.05% | Slightly turbid solution | Turbid solution |
| Malic Acid) | | |
| E | Pass (4) | Pass (3) |
| (0.10% | Clear solution | Minimally turbid |
| Malic Acid) | | solution |
| F | Pass (4) | Pass (4) |
| (0.30% | Clear solution | Clear solution |
| Malic Acid) | | |
| G | Pass (4) | Pass (4) |
| (0.40% | Clear solution | Clear solution |
| Malic Acid) | | |
| H | Pass (4) | Pass (4) |
| (0.50% | Clear solution | Clear solution |
| Malic Acid) | | |

What is claimed is:

1. An oral care mouth rinse composition comprising
(i) at least one source of ionic tin and
(ii) at least one aliphatic di- or tri-carboxylic acid in free or salt form wherein the at least one aliphatic di- or tri-carboxylic acid in free or salt form is present in an amount of from 0.05 to 0.30 weight % based on the total weight of the oral care composition.

2. The oral care mouth rinse composition of claim 1 comprising 0.10 to 0.25 weight % aliphatic di- or tri-carboxylic acid in free or salt form.

3. The oral care mouth rinse composition of claim 1 wherein the at leak one aliphatic di- or tri-carboxylic acid in free or salt form comprises a C₃ to C₇ aliphatic di-carboxylic acid in free or salt form.

4. The oral care mouth rinse composition of claim 1 comprising 0.10 to 0.25 weight % malic acid.

5. The oral care composition of claim 1 wherein the source of ionic tin is selected from the group comprising stannous ion sources, stannic ion sources and combinations thereof.

6. The oral care mouth rinse composition of claim 1 wherein the source of ionic tin is selected from the group comprising stannous fluoride, stannous chloride, stannic fluoride, stannic chloride, stannic acetate, stannous acetate and combinations thereof.

7. The oral care mouth rinse composition of claim 1 wherein the composition comprises at least one stannous ion source.

8. The oral care mouth rinse composition of claim 1 wherein the source of ionic tin is selected from the group comprising stannous fluoride, stannous chloride, stannous acetate and combinations thereof.

9. The oral care mouth rinse composition of claim 1 wherein the concentration of ionic tin is from 0.01 to 0.10 weight % based on the total weight of the oral care mouth rinse composition.

10. The oral care mouth rinse composition of claim 1 wherein the source of ionic tin comprises stannous fluoride.

11. The oral care mouth rinse composition of claim further comprising a non-stannous fluoride ion source.

12. The oral care mouth rinse composition of claim 1 comprising sodium fluoride, potassium fluoride, sodium monofluorophosphate, sodium fluorosilicate, ammonium fluorosilicate, amine fluoride, ammonium fluoride and combinations thereof.

13. The oral care mouth rinse composition of claim 1 comprising sodium fluoride or amine fluoride.

14. The oral care mouth rinse composition of claim 1 comprising amine fluoride or sodium fluoride in an amount corresponding to 45 to 1500 ppm fluoride based on the total weight of the composition.

15. The oral care mouth rinse composition of claim 1 comprising 0.06 to 0.33 weight % amine fluoride or 0.01 to 0.33 weight % sodium fluoride or a combination thereof based on the total weight of the composition.

16. The oral care mouth rinse composition of claim 1 comprising 0.10 to 0.25 weight % malic acid, 0.04 to 0.08 weight % stannous fluoride and 0.10 to 0.25 weight % N,N,N′-tris(2-hydroxyethyl)-N′-octadecyl-1,3-diaminopropane dihydrofluoride.

17. A method to
(i) reduce or inhibit formation of dental caries,
(ii) reduce, repair or inhibit pre-carious lesions of the enamel,
(iii) reduce or inhibit demineralization and promote remineralization of the teeth,
(iv) reduce hypersensitivity of the teeth,
(v) reduce or inhibit gingivitis,
(vi) promote healing of sores or cuts in the oral cavity,
(vii) reduce levels of acid producing bacteria,
(viii) reduce or inhibit microbial biofilm formation in the oral cavity,
(ix) reduce or inhibit plaque formation in the oral cavity,
(x) promote systemic health, or
(xi) clean teeth and oral cavity,
comprising applying an effective amount of an oral care mouth rinse composition according to claim 1 to the oral cavity of a subject in need thereof.

18. An oral care mouth rinse composition according to claim 1 for use in a method to reduce or inhibit formation of dental caries,
(i) reduce, repair or inhibit pre-carious lesions of the enamel,
(ii) reduce or inhibit demineralization and promote remineralization of the teeth,
(iii) reduce hypersensitivity of the teeth,
(iv) reduce or inhibit gingivitis,
(v) promote healing of sores or cuts in the oral cavity,
(vi) reduce levels of acid producing bacteria,
(vii) reduce or inhibit microbial biofilm formation in the oral cavity,
(viii) reduce or inhibit plaque formation in the oral cavity,
(ix) promote systemic health, or
(x) clean teeth and oral cavity.

19. A method of stabilizing ionic tin in an oral care mouth rinse composition comprising at least one source of ionic tin, wherein the method comprises formulating the oral care mouth rinse composition to comprise at least one aliphatic di- or tri-carboxylic acid in free or salt form in an amount of from 0.05 to 0.30 weight % based on the total weight of the oral care composition.

20. A method of reducing staining of surfaces in the oral cavity resulting from use of an oral care mouth rinse comprising at least one source of ionic tin, wherein the method comprises formulating the oral care mouth rinse composition to comprise at least one aliphatic di- or tri-carboxylic acid in free or salt form in an amount of from 0.05 to 0.30 weight % based on the total weight of the oral care composition.
